(19) United States
(12) Patent Application Publication
(10) Pub. No.: US 2014/0142410 A1
(43) Pub. Date: May 22, 2014

(54) Nerve Stimulation Electrodes

(71) Applicant: STROHL MEDICAL, Norwell, MA (US)

(72) Inventors: Michael K. Erb, Brighton, MA (US); Clair L. Strohl, Norfolk, MA (US)

(73) Assignee: STROHL MEDICAL, Norwell, MA (US)

(21) Appl. No.: 13/681,073

(22) Filed: Nov. 19, 2012

(51) Int. Cl. A61B 5/0484; A61B 5/0478

(52) U.S. Cl. CPC A61B 5/0484; A61B 5/0478
USPC 600/383; 600/395; 600/396; 600/391; 600/392; 600/384

(57) ABSTRACT

Described herein are nerve stimulating electrodes for use in evoked potential electrophysiological recording procedures and associated methods for use thereof. In one embodiment, a stimulating electrode assembly is provided that includes a non-conductive substrate, a first electrode mounted on the non-conductive substrate, and a second electrode mounted on the non-conductive substrate. The first and second electrodes are spaced apart along a longitudinal length of the assembly and extend along a lateral width of the assembly such that the electrode can be positioned at a plurality of laterally offsetting positions relative to a nerve extending longitudinally between the first and second electrodes while remaining in contact with the nerve.

### FIELD

This application relates generally to electrophysiological recording technology and, more particularly, to stimulating electrodes that can be used to produce evoked potential responses that can be measured in a patient's brain.

### BACKGROUND

Electrophysiological recording technologies measure the electrical activity present in a patient's body. Certain types of electrophysiological recording technologies, such as electroencephalography (EEG) and electromyography (EMG) can measure “evoked potential” (EP) electrical signals created by the body in response to a stimulus. In the case of EEG, for example, detection electrodes attached to a patient's scalp can monitor an evoked potential created in the patient's brain in response to the stimulation of a nerve elsewhere in the body.

Evoked potential electroencephalography can be utilized in a variety of manners. For example, by stimulating nerves on both sides of a patient's body and monitoring the electrical signals subsequently created in the patient's brain, determinations can be made regarding the presence of ischemia (inadequate blood supply) in the brain. Such a determination can be utilized to support accurate diagnosis and treatment of, for example, a progressively developing stroke in a patient.

Despite the usefulness of such a procedure, EP monitoring technologies such as EEG are not routinely used due to the difficulty of placing the stimulating electrodes properly. Proper placement requires accurately positioning stimulating electrodes over a nerve structure as well as positioning sensing electrodes over a patient's scalp to detect responses in the appropriate area of the brain. If the stimulating electrodes are not properly placed to provide uniform and consistent stimulation on both sides of the body, the effectiveness of the procedure for diagnosis can be reduced. This is because uneven stimulation on either side of the body can produce uneven evoked potential signals in the patient's brain that may be indicative of ischemia or other problems.

Accordingly, there is a need for improved stimulating electrodes for use in evoked potential electrophysiological recording procedures.

### SUMMARY

The present invention generally provides a stimulating electrode for use in evoked potential electrophysiological recording procedures. One advantage is that the devices and methods described herein permit technicians with lower levels of training to accurately place stimulating electrodes over a nerve structure to produce consistent and uniform stimulation of the structure. The devices disclosed herein can include a number of features. For example, the electrodes disclosed herein can be bipolar to permit the transmission of stimulating electrical signals, and can have a large functional area (i.e., electrical contact surface area) that allows a technician a margin of error when placing the electrode on a patient's skin. The electrodes can also or alternatively be formed of low impedance materials to minimize the strength of the electrical signal required to stimulate the nerve structure. Furthermore, the electrodes can be configured to securely attach to a patient's skin such that they are “hands-free” for the technician administering the procedure, and the electrodes can be disposable to avoid the time and costs associated with cleaning, testing, and sterilizing them for reuse. These and other features of the electrodes disclosed herein are discussed in more detail below.

In one aspect, a stimulating electrode assembly is provided that includes a non-conductive substrate as well as a first electrode and a second electrode that are mounted on the non-conductive substrate. The first electrode includes a first porous pad having an elongate shape, e.g., a substantially rectangular shape, and is impregnated with a conductive gel. The second electrode can be spaced a predetermined distance apart from the first electrode and can include a second porous pad having an elongate shape, e.g., a substantially rectangular shape. The second electrode can also be impregnated with a conductive gel. Furthermore, the first and second electrodes can be configured to deliver stimulating energy through the conductive gel.

The stimulating electrode assembly can have a number of variations and additional features, all of which are considered within the scope of the present invention. For example, the predetermined distance between the first and second electrodes can be varied according to the nerve being targeted, the size of the patient, etc. In certain embodiments, the predetermined distance between the first and second electrodes can be in the range of about 10 mm to about 30 mm. In other embodiments, the predetermined distance can be about 22 mm.

In addition, the first and second electrodes can have a number of configurations. For example, in some embodiments the first and second porous pads can each have a length in the range of about 25 mm to about 40 mm. In other embodiments, the non-conductive substrate can have first and second opposed surfaces, an adhesive can be disposed on the first surface, and the first and second porous pads can be mounted on the first surface.

In other embodiments, the first electrode can be configured as a cathode and the second electrode can be configured as an anode. In still other embodiments, the first and second electrodes can each include a conductive button disposed between the non-conductive substrate and the porous pad for delivering energy to the conductive gel in the porous pad. The conductive button can be formed from a variety of materials but, in some embodiments, the conductive button can be formed of silver/silver chloride. In certain embodiments, the first and second electrodes can each include a conductive sheet disposed between the non-conductive substrate and the porous pad for delivering energy to the conductive gel in the porous pad. The conductive sheet can, in some embodiments, also be formed of silver/silver chloride. In still other embodiments, the impedance of each of the first and second electrodes can be in a range of about 0 Ohms to about 10,000 Ohms.

The first and second porous pads of the assembly can be formed from a number of different materials. In some embodiments, the first and second porous pads can each be formed of foam and, in certain embodiments, the first and second porous pads can be formed of reticulated foam. The foams used to form the first and second porous pads can have a variety of different porosities. In some embodiments, for example, the first and second porous pads can each be formed of foam having a porosity in a range of about 10 pores per inch to about 90 pores per inch.

In addition, in certain embodiments the assembly can include a first lead wire coupled to and extending from the first electrode, and a second lead wire coupled to and extending from the second electrode. The first and second lead wires can be formed from a variety of conductive materials and can have any length desired. The first and second lead wires can be used to connect the first and second electrodes to a power source to provide stimulating energy.

In another aspect, a stimulating electrode assembly is provided that includes a substrate having an adhesive disposed on a surface thereof for adhering the substrate to a tissue surface. The assembly further includes a first electrode disposed on the substrate, the first electrode including a first porous pad having a width greater than a length thereof. The assembly also includes a second electrode disposed on the substrate and spaced a distance apart from the first electrode, the second electrode including a second porous pad having a width greater than a length thereof. The first and second porous pads can have a surface area and a shape effective to allow targeting of a nerve with electrical pulses when paced such that a nerve extends between the first and second porous pads at any position along the width thereof.

In some embodiments, the first and second porous pads can each have an elongate rectangular shape and extend substantially parallel to each other. The distance between the first and second porous pads can vary but, in some embodiments, the distance between the porous pads can be about 22 mm.

Each of the first and second electrodes can have a variety of assembly configurations. In certain embodiments, for example, the first and second electrodes can include a conductive button positioned between the substrate and the porous pad for delivering energy to the porous pad. In other embodiments, the first and second porous pads can each include a conductive sheet positioned between the substrate and the porous pad for delivering energy to the porous pad. Still further, in some embodiments, the first and second porous pads can be impregnated with a conductive gel.

In another aspect, a method for stimulating a nerve is provided that includes affixing a substrate to a patient's skin surface to position first and second electrodes mounted on the substrate in contact with the skin surface. The electrodes can be positioned such that the nerve extends longitudinally across the first and second electrodes, and the first and second electrodes can each have a width extending laterally such that the electrodes can be positioned at a plurality of laterally offset positions relative to the nerve extending longitudinally therebetween while remaining in contact with the nerve. The method can further include delivering electrical energy to the nerve through the first and second electrodes. In some embodiments, the method can further include detecting a signal generated in the patient's brain in response to the delivered electrical energy using a plurality of sensors positioned around the patient's head.

In still another aspect, a method for applying a stimulating electrode assembly is provided that includes peeling one of a plurality of electrode assemblies away from a ribbon wire extending between the plurality of electrode assemblies and a connector. The method can further include affixing the one of the plurality of electrode assemblies adjacent to a nerve to be stimulated in a patient's body, and activating an energy source to deliver a targeted electrical signal to the nerve. The method can also include repeating the steps of peeling, affixing, and activating for at least one additional electrode of the plurality of electrode assemblies. In some embodiments, the method can also include coupling the connector to a source of electrical energy.

The electrode assemblies can be affixed to a patient's skin at a variety of locations. In certain embodiments, the one of the plurality of electrode assemblies can be affixed to a patient's wrist to stimulate the median nerve upon delivery of the electrical signal. In other embodiments, the one of the plurality of electrode assemblies can be affixed to the patient's knee to stimulate the common peroneal nerve upon delivery of the electrical signal.

### BRIEF DESCRIPTION OF THE DRAWINGS

The aspects and embodiments of the invention described above will be more fully understood from the following detailed description taken in conjunction with the accompanying drawings, in which:

FIG. 1 is a bottom view of an embodiment of a stimulating electrode assembly;

FIG. 2 is a top view of the stimulating electrode assembly of FIG. 1;

FIG. 3 is a side view of the stimulating electrode assembly of FIG. 1;

FIG. 4 is a side exploded view of the stimulating electrode assembly of FIG. 1;

FIG. 5 is a bottom view of one embodiment of an electrode of a stimulating electrode assembly;

FIG. 6 is a diagram depicting the median nerve extending through a patient's arm;

FIG. 7 is a diagram depicting the common peroneal nerve extending through a patient's leg;

FIG. 8 is a diagram depicting exemplary placement of a stimulating electrode assembly with respect to the median nerve; and

FIG. 9 is a diagram depicting several sets of stimulating electrode assemblies connected by a ribbon cable of lead wires.

### DETAILED DESCRIPTION

Certain exemplary embodiments will now be described to provide an overall understanding of the principles of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

In one aspect, the present invention relates to stimulating electrode assemblies for use in evoked potential electrophysiological recording procedures, though the invention can be broadly applied to any area that utilizes a bipolar stimulating electrode. The electrode assemblies described herein can include a number of features that enable the electrodes to deliver uniform and consistent stimulation signals to a patient. Exemplary features that can enable this functionality include construction using low impedance materials, electrode surface areas and geometric designs that allow a margin of error for placement, and adhesive backings to allow hands-free operation by a technician. In addition, the electrodes described herein can be disposable and can include integral lead wires, thereby avoiding the time delay and performance complications that can be associated with repeated assembly prior to a procedure, as well as the costs associated with cleaning, testing, and sterilization between procedures.

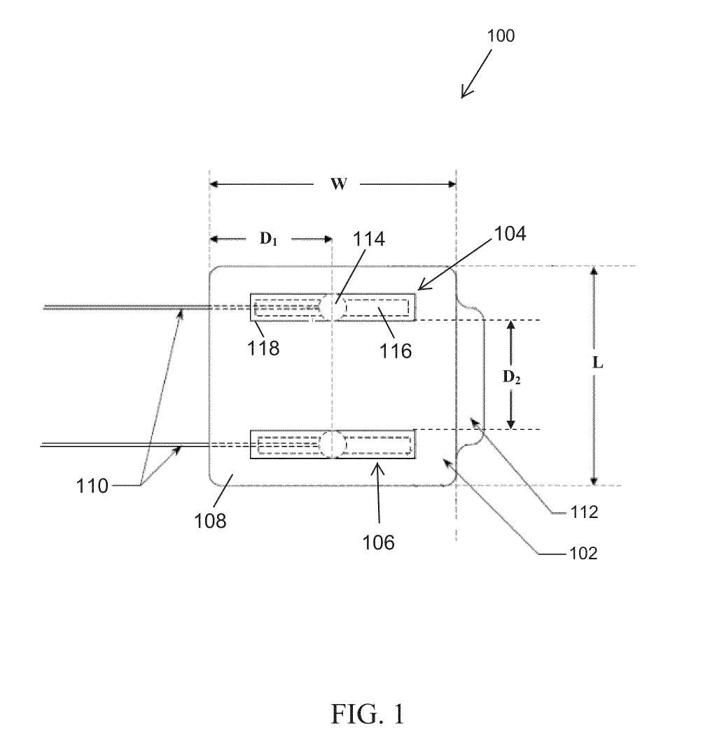

FIG. 1 illustrates a bottom view of one embodiment of a stimulating electrode assembly according to the teachings of the invention. The electrode assembly 100 can include a substrate 102 having mounted thereon a first electrode 104 and a second electrode 106. The first and second electrodes can be mounted on a first surface 108 of the substrate 102, and can be coupled to electrical lead wires 110 that extend from an opposing, second surface of the substrate (not shown).

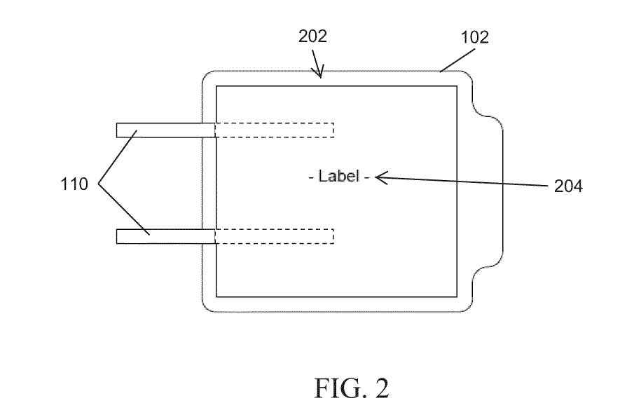

FIG. 2 illustrates a top view of the stimulating electrode assembly of FIG. 1. Visible in FIG. 2 is the opposing, second surface 202 of the substrate 102. The second surface 202 can have disposed thereon a label 204 that can include, for example, instructions regarding the placement of the electrode assembly 100 on a patient's skin. The electrical lead wires 110 can extend along the second surface 202 such that they are sandwiched between the second surface and the label 204, or they can extend through a hole formed in the label 204 and rest on top of it.

Referring back to FIG. 1, the substrate 102 can have a variety of shapes and dimensions, including longitudinal lengths and lateral widths, as shown by reference letters L and W in the figure. In some embodiments, for example, the length L and width W of the substrate 102 can be in a range of about 3 cm to about 7 cm. In certain embodiments, for example, the width W can be about 5.1 cm and the width W can be about 5.7 cm. The substrate 102 can be formed from a variety of materials and, in some embodiments, can be formed from a foam material. In other embodiments, the substrate 102 can be formed from a sponge or felt material. The material used to form the substrate 102 can also be non-conductive to prevent electrical shorts from forming between the first and second electrodes 104, 106 disposed thereon. The first surface 108 can be configured to contact a patient's skin such that the first and second electrodes 104, 106 come into contact with the skin. To aid in placement, the first surface 108 can have an adhesive disposed thereon over all or a part of the surface's area to aid in retaining the position of the electrode assembly on a patient's skin. The use of an adhesive can allow the hands-free operation of the electrode assembly, as a technician need not hold the assembly in place over an area of a patient's skin. Still further, the substrate 102 can include a tab 112 or other feature that can facilitate the ease of grasping for removal or repositioning of the electrode assembly. For example, in some embodiments, the tab 112 can be a portion of the substrate 102 that does not have adhesive disposed thereon such that the tab can be easily grasped by a technician to remove or reposition the electrode assembly. In some embodiments, the substrate 102 can be manufactured with adhesive covering the entire first surface 108 (including the tab 112), but a protective backing (not shown) can be placed over the adhesive to protect from inadvertent attachment prior to use. In such an embodiment, the protective backing can include a perforation or cutout such that the protective backing remains over the tab 112 when the backing is removed from the remainder of the substrate.

As mentioned above, the first and second electrodes 104, 106 can be mounted on the first surface 108 of the substrate 102. Each of the first and second electrodes 104, 106 can include a conductive element 114, a conductive sheet 116, and a porous pad 118. The button 114 and sheet 116 can be formed from a variety of conductive materials, including metallic materials, conductive polymers, and conductive composites. In some embodiments, these components can be formed from Silver/Silver Chloride (Ag/AgCl). Other exemplary materials can include gold, tin, and steel. The button 114 can be in electrical contact with the lead wire 110, and can also be in electrical contact with the sheet 116 and the porous pad 118. In order to efficiently conduct electrical energy from the lead wire 110 into the patient's body, the porous pad 118 can be impregnated with a conductive gel or another suitable substance, such as a liquid. The conductive gel can be a hydrogel or a wet gel. In some embodiments, a wet gel can be useful because it can have a lower impedance than a hydrogel. The porous pad 118 can be formed from a variety of materials and, in some embodiments, can be formed of foam, such as reticulated foam. In certain embodiments, reticulated foams can be formed from polyurethane, polyethylene, polypropylene, nylon or polyester. In addition, the porous pad 118 can also be formed from a sponge or felt material (similar to the substrate 102 discussed above), as these materials can also hold the conductive, low impedance gel. Sponges and felts can, in some embodiments, be made from natural materials such as cotton and wool. In some embodiments, the porous pad can be formed of reticulated foam having a porosity in a range of about 10 pores per inch to about 90 pores per inch.

The first and second electrodes 104, 106 can be shaped and positioned on the substrate 102 so as to provide uniquely uniform and consistent electrical stimulation to a patient's nerves despite irregularities in positioning by medical technicians administering a procedure. In some embodiments, as shown in FIG. 1, the first and second electrodes 104, 106 can have an elongate rectangular shape and extend substantially parallel to each other. The elongate rectangular shape of each electrode 104, 106 can be achieved, for example, by forming the porous pad 118 of each electrode into an elongate rectangular shape. The first and second electrodes 104, 106 can be positioned on the substrate 102 such that they each extend along a lateral width W of the substrate and are separated from one another along a longitudinal length L of the substrate.

For example, in some embodiments, the first and second electrodes 104, 106 can be centered a distance D₁ from a lateral edge of the substrate 102 and extend along the lateral width W of the substrate. In certain embodiments, the distance D₁ can be about half the width W of the substrate, thereby centering the first and second electrodes on the substrate in the lateral direction. By way of example, in some embodiments, the distance D₁ can be in a range of about 20 mm to about 30 mm and, in certain embodiments, the distance D₁ can be about 28 mm.

In addition, the first and second electrodes 104, 106 can be separated from one another by a distance D₂ extending along the longitudinal length L of the substrate 102. The distance D₂ can be selected such that efficient stimulation of a patient's nerve is possible without allowing any electrical short to develop between the two electrodes 104, 106. In some embodiments, the distance D₂ can be in a range of about 10 mm to about 30 mm and, in some embodiments, the distance D₂ can be about 22 mm. With the distance D₂ selected to prevent electrical shorting between the first and second electrodes 104, 106, the electrode assembly 100 can be configured as a bipolar stimulating electrode wherein, for example, the first electrode 104 can be configured as a cathode and the second electrode 106 can be configured as an anode.

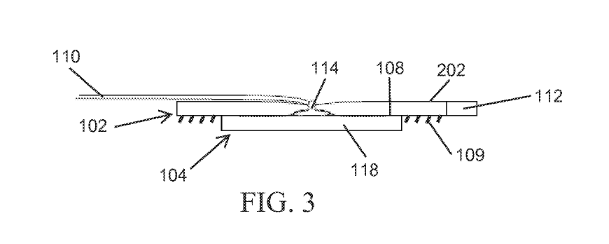

FIG. 3 illustrates a side view of the electrode assembly 100 of FIGS. 1 and 2. This alternate view shows both the first surface 108 and the second surface 202 of the substrate 102, as well as the tab 112 extending from a side of the substrate 102. Also shown is the first electrode 104, which can include a conductive button 114 that is disposed between the non-conductive substrate 102 and the porous pad 118. The button 114 can extend through the substrate 102 from the first surface 108 to the second surface 202 and can be electrically coupled to a lead wire 110 near the second surface. The conductive button 114 can also be electrically coupled to the porous pad 118 of the first electrode 104 through direct contact and/or through the conductive sheet 116 (not visible). In addition, the label 204 (not visible) can be disposed on the second surface 202.

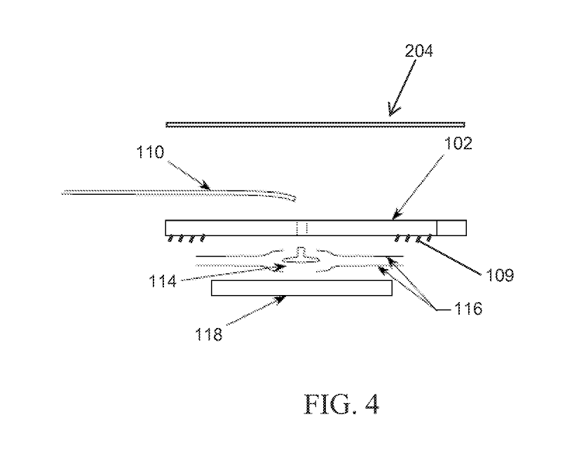

FIG. 4 depicts an exploded side view of the electrode assembly 100 of FIGS. 1-3. In this view the conductive sheet 116 is shown disposed between the non-conductive substrate 102 and the porous pad 118. The sheet 116 can include a single piece of material or, as shown in the figure, can include two or more pieces of material. Each piece of material can be shaped to interface with the conductive button 114 so as to conduct electrical energy from the conductive button into the conductive gel impregnated in the porous pad 118. In some embodiments, the conductive button 114 and conductive sheet 116 can be formed as a single element, e.g., a conductive sheet having a raised structure formed thereon that can extend through the non-conductive substrate 102. Also visible in FIG. 4 is the label 204 that can be disposed on the surface of the non-conductive substrate 102 opposite from the first and second electrodes 104, 106. As mentioned above, the label can be applied so as to sandwich the lead wire 110 between the label 204 and the non-conductive substrate 102, or it can include an opening formed therein that allows the lead wire 110 to extend through the label 204 and rest on top of it.

FIGS. 3 and 4 illustrate one exemplary embodiment of the construction of the first and second electrodes 104, 106. Other configurations are also possible. For example, in some embodiments, the first and second electrodes can be silkscreened on a non-conductive substrate material using, for example, conductive inks or other materials. Such an electrode formed on the substrate can be coupled directly to a porous pad, or in still other embodiments, a conductive sheet can be included between the porous pad and the silkscreened electrode.

As mentioned above, one advantageous feature of the stimulating nerve electrodes described herein is the margin of error that is provided for inaccurate placement of the electrode on a patient's body. This margin of error allows a lesser-trained technician to administer the procedure in a reduced amount of time because uniform and consistent electrical signals can be delivered to a patient's nerve structure despite imperfect placement of the stimulating electrodes.

This margin of error is provided in part by the shape and positioning of the electrodes in an electrode assembly that is attached to the patient. As shown in FIG. 1, the first and second electrodes 104, 106 can be separated by a distance along a longitudinal length L of the electrode assembly 100, and they can be configured to extend along a lateral width W such that each electrode has an increased functional surface area to contact a nerve structure. This lateral extension can allow the electrode assembly 100 to be placed in a variety of laterally offset positions while still covering the nerve structure such that electrical stimulation can be efficiently accomplished.

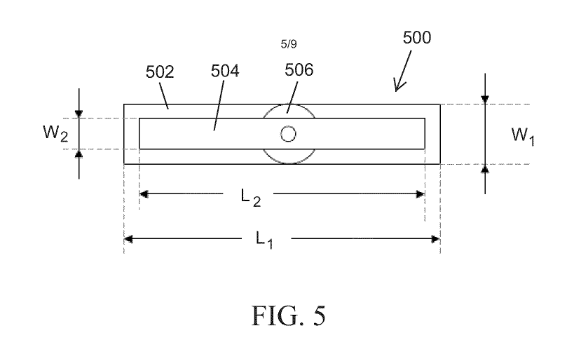

In some embodiments, each electrode in an electrode assembly 100 can have an elongate shape, such as a substantially rectangular shape, with a length in a range of about 25 mm to about 40 mm. FIG. 5 illustrates one embodiment of an electrode 500 that includes a porous pad 502, conductive sheet 504, and conductive button 506. In the embodiment shown in FIG. 5, the electrode 500 can have an overall length L₁ of about 3.8 cm and an overall width W₁ of about 1 cm. The overall length and width can be determined, for example, by the dimensions of the porous pad 502. The conductive sheet 504 can have smaller dimensions. For example, the conductive sheet can have a length L₂ of about 3 cm and a width W₂ of about 6 mm. In other embodiments, slightly smaller size electrodes can be employed having an overall length L₁ of about 3.2 cm and the same overall width W₁ of about 1 cm. In such an embodiment, the conductive sheet can have a length L₂ of about 2.4 cm and a same width W₂ of about 6 mm. In still another embodiment, the overall length L₁ of the electrode can be about 2.5 cm with a same overall width W₁ of about 1 cm. In this embodiment, the length L₂ of the conductive sheet can be about 1.8 cm with a same width W₂ of about 6 mm. By varying the lengths of the electrodes used, an electrode assembly 100 can be created having a low impedance while allowing for a margin of error in placement of the electrode on a patient's body.

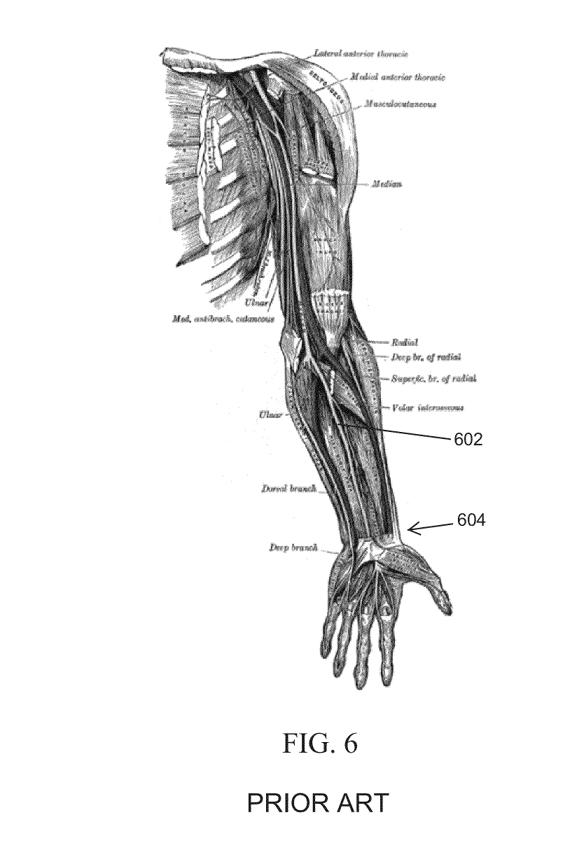

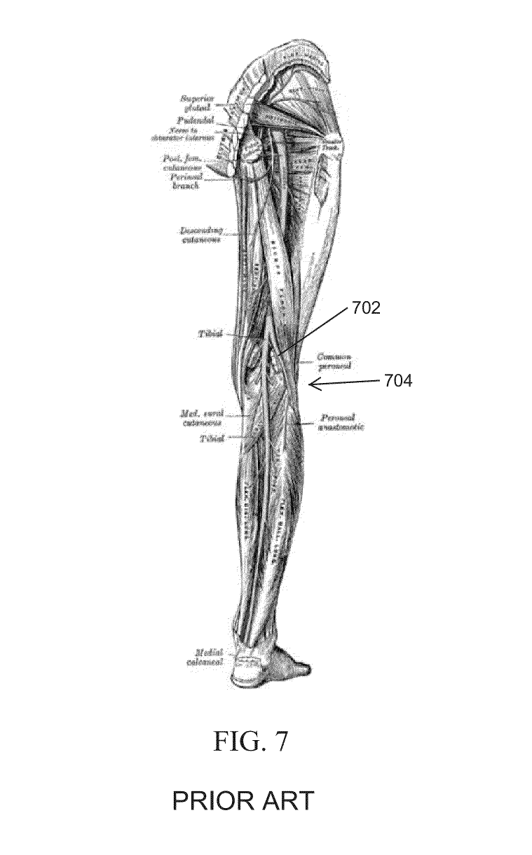

The stimulating nerve electrodes and electrode assemblies described herein can be utilized in a variety of procedures and are suitable for placement at a variety of locations of a patient's body. Two common locations for placement during evoked potential electroencephalography are above the median nerve in the arm and the common peroneal nerve in the leg. FIG. 6 depicts the human arm and shows the location of the median nerve 602 extending along the length of the arm. FIG. 7 depicts the human leg and shows the location of the common peroneal nerve 702 extending along the length of the leg.

In the arm, the median nerve runs closest to the surface of the skin in the area of a patient's wrist 604 on the palm-side. As a result, stimulating nerve electrodes are typically placed in this area to deliver electrical signals to the median nerve. In the leg, the common peroneal nerve is closest to the surface of the skin near the backside of a patient's knee 704. Accordingly, stimulating nerve electrodes are typically placed in this area to deliver electrical signals to the common peroneal nerve. By placing bipolar electrode assemblies in these two locations on each side of a patient's body (i.e., four total stimulating electrode assemblies) and delivering stimulating electrical signals therethrough, differences in the evoked potentials developed in the patient's brain can be monitored and analyzed to detect the presence and extent of ischemia in the brain. As a result of the fact that differential brain function is central to diagnosis, uniform and consistent stimulation on both sides of a patient's body is essential to prevent false positive detection. More information on using stimulating electrical signals to diagnose medical conditions can be found in U.S. Pat. No. 7,024,238 to Bergethon, entitled “Detecting Ischemia,” which is hereby incorporated by reference in its entirety.

As mentioned above, in administering evoked potential electroencephalography procedures, proper placement of the electrode assemblies over the nerve structures being targeted is essential for accurate diagnosis. In the case of the median and common peroneal nerves, electrode assemblies must be placed at particular areas where the nerve structures are located close the surface of the patient's skin. These areas include the palm-side of a patient's wrist for the median nerve, and the backside of the patient's knee for the common peroneal nerve. At other more proximal and distal locations along the arm and leg, the nerve is located deeper below the surface of the skin and cannot be efficiently stimulated using an electrode placed on the patient's skin.

Still further, administering technicians often have only a single chance to successfully place a stimulating electrode assembly due to the conductive gel that is often used in such assemblies. If a technician incorrectly places an electrode assembly and subsequently slides it across a patient's skin to correct the problem, the conductive gel can be smeared across the patient's skin and can produce an electrical short by providing a low-resistance electrical path between the anode and cathode of an electrode assembly.

The stimulating electrode assemblies described herein can include features that can compensate for errors in placement of the electrode assemblies. These features can include, for example, electrodes that have an elongate shape extending in a particular direction such that the assembly can be placed in a variety of positions while still remaining in contact with (e.g., being positioned directly over) a nerve structure. Such a stimulating electrode assembly can allow a lesser-trained technician to successfully administer electroencephalography, thereby increasing the number of eligible administering technicians and the availability of the procedure to patients.

In one exemplary embodiment, a technician can affix a substrate to a patient's skin surface so as to position first and second electrodes mounted on the substrate in contact with the skin surface of the patient. The first and second electrodes can be positioned such that a nerve of the patient extends longitudinally across the first and second electrodes. Further, the first and second electrodes can have a width extending laterally such that the electrodes can be positioned at a plurality of laterally offset positions relative to the nerve while remaining in contact with the nerve.

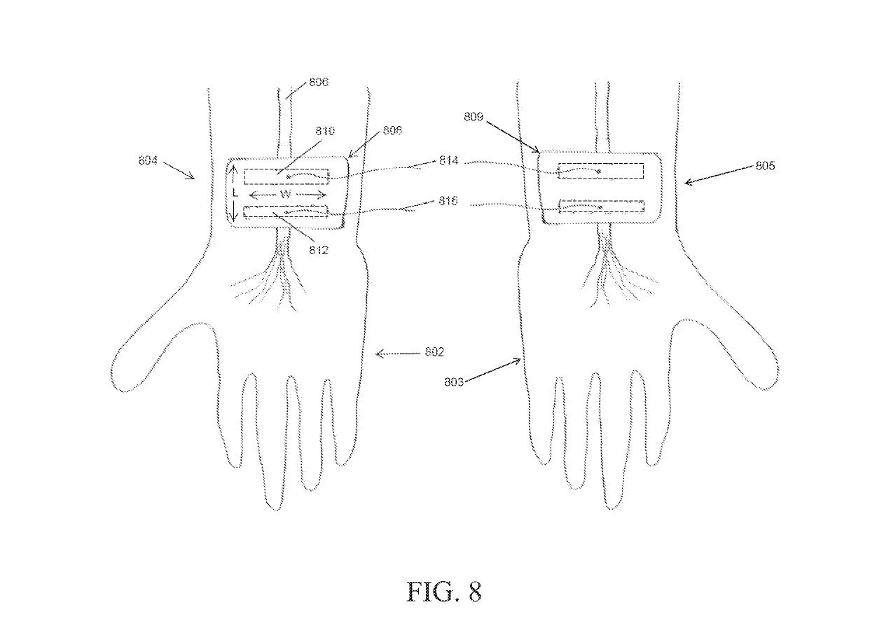

An example of the placement of such first and second electrodes is depicted in FIG. 8. The figure shows a top-view of the palm-side of a patient's hand 802 and wrist 804. Also illustrated is the median nerve 806 extending longitudinally down the patient's arm, through the wrist 804, and into the hand 802. An electrode assembly 808 can be positioned over the wrist 804 to stimulate the median nerve 806 using first and second electrodes 810, 812. The first and second electrodes 810, 812 can each have an elongate shape extending along a lateral width W and the first and second electrodes can be separated along a longitudinal length L of the assembly 808. The advantage of this configuration is clear in the figure—the stimulating electrode assembly 808 can be positioned at several laterally offset positions (i.e., positions offset to the left or right from the position shown along the lateral width W) while still remaining in contact with (e.g., being positioned directly over) the median nerve 806. The first and second electrodes 810, 812 can be electrically coupled to first and second lead wires 814, 816, respectively, to receive electrical signals from a power source. A variety of types and strengths of electrical signals can be delivered from the power source. In one embodiment, the electrical signals delivered from the power source can be in the form of a square wave pulse train having a pulse repetition rate in a range of about 0.1 Hz to about 15 Hz, a pulse duration in a range of about 0.050 milliseconds to about 1.000 milliseconds, and an amplitude in a range of about 0 to about 100 milliamperes.

After successfully positioning the stimulating electrode assembly 808, the administering technician can repeat the procedure on the opposite side of the body using a second stimulating electrode assembly, and then repeat the procedure twice more to place two additional stimulating electrode assemblies over the common peroneal nerve on each side of the patient's body. Sensing electrodes placed over the patient's scalp can then be used to measure the evoked potential electrical signals generated in the patient's brain in response to electrical signals delivered through the four stimulating electrode assemblies.

The stimulating electrode assemblies described herein can also utilize a ribbon cable that provides four electrode assemblies in a single bundle, as well as permanent electrical connections between each strand of the cable and one of the electrodes in an electrode assembly. Such a configuration can be designed for a single use as a disposable component. For example, the ribbon cable can reduce the cluttered appearance of the testing apparatus and eliminate problems with wires becoming tangled or crossed during a procedure. In addition, the use of permanent electrical connections between each strand of wire and electrode in an electrode assembly can provide high quality and low impedance electrical coupling between a power source and the electrode. In other embodiments, however, multiple separate wires can also be used to connect each electrode to a power source.

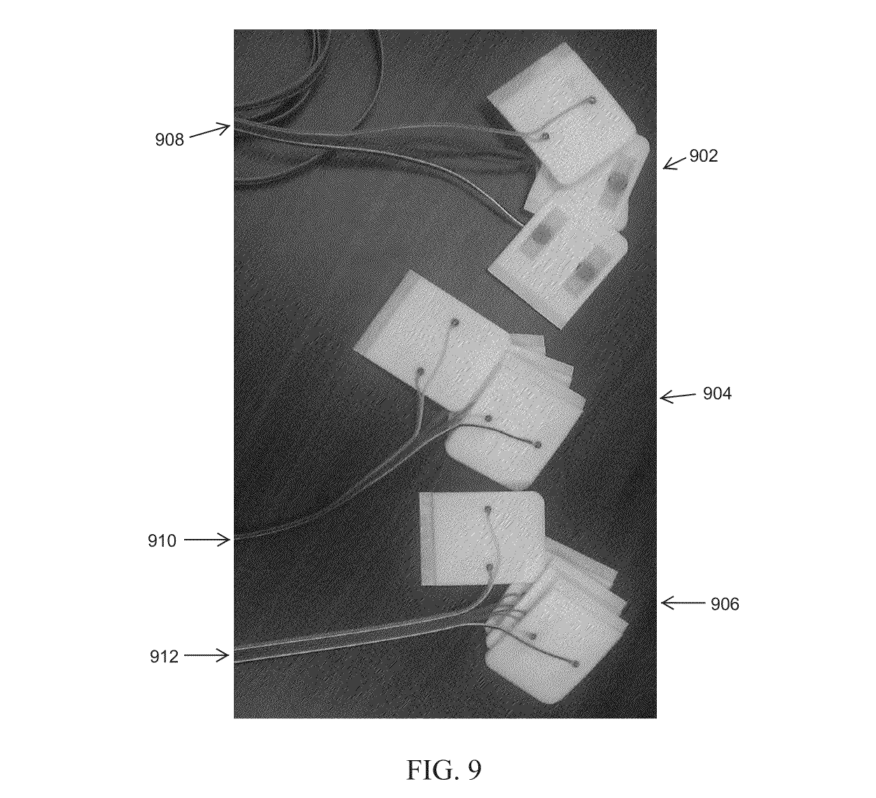

FIG. 9 depicts an exemplary embodiment of a ribbon cable in combination with sets of electrode assemblies. In particular, FIG. 9 depicts three sets 902, 904, 906 of four electrode assemblies (e.g., configured for placement over a nerve structure on both sides of a patient's body in an upper and lower extremity) that each includes a ribbon cable 908, 910, 912 connecting the electrodes from each electrode assembly to a power source (not shown). In some embodiments, the sets of electrode assemblies can arrive in the configuration shown by sets 904 and 906—that is, with the ribbon cable extending from a proximal end that is configured to interface with a power source to a distal end where the strands of the cable break away for a short distance to connect to each of the electrode assemblies. When a technician sets up for an electroencephalography procedure, the electrode assembly set and ribbon cable can be unpackaged, and the technician can peel each electrode assembly away from the others as he or she places the electrode assembly on a patient's body. Such a peeling action can separate the two strands connected to any electrode assembly away from the other strands. The ribbon can be peeled away for as much of the length of the ribbon cable as is required to reach the placement site, and can remain connected to the other strands of the ribbon cable at a more proximal location, as shown by set 902 in FIG. 9.

The ribboned electrode assembly sets can have a further advantage in that the proximal end of the ribbon cables 908, 910, 912 can remain ribboned together to reduce clutter when connecting to a power source (not shown). For example, the proximal end of the ribbon cable can include a connector element configured to allow the connection of each strand in the ribbon cable with only one action by a technician. Alternatively, a technician or other user can separate the strands of the ribbon cable at the proximal end in addition to the distal end to facilitate connecting the electrode assemblies to a power source. In such an embodiment, strands can remain ribboned together in a middle portion between the proximal and distal ends of the ribbon cable, which maintains a reduced amount of wire clutter. One advantage of including a connector element at the proximal end of the ribbon cable can be the reduction of technician error by configuring the connector element to couple with the power source in only a single orientation. Such a connector, coupled with a designation of the intended area of the body for each electrode assembly using, for example, the label 204 on each electrode assembly, can reduce the incidence of a technician crossing wires and connecting, for example, the electrode on a patient's wrist to an outlet on a power source designated for the electrode on the patient's knee.

In the embodiments discussed above, sets of four stimulating electrode assemblies have been described. However, in some embodiments, more or fewer electrode assemblies can be included. In some embodiments, for example, one or more additional sensing electrodes can be included as a separate component or as an additional electrode and wire strand that is part of the ribbon cable and electrode assembly set. This additional sensing electrode can be placed by a technician at an intermediate point between the patient's scalp and any of the stimulating electrode assemblies (e.g., near a patient's neck or on their upper arm). In such a position, the sensing electrode can be used to detect the propagation of a stimulating electrical signal along the nerve chain extending from the extremity into the patient's brain. Sensing the propagation of a stimulating electrical signal in this manner can aid in troubleshooting the positioning of the electrode assemblies and tuning of the power source to prevent false negatives during an electroencephalography procedure.

The use of an intermediate sensing electrode can also help reduce the tendency for technicians to increase the stimulating signal amplitude in the event a suitable evoked potential response is not observed in the patient's brain. The sensing electrode does so by detecting the presence of the stimulating electrode closer to the source, which can allow a determination to be made regarding the quality of stimulus being delivered at the stimulating electrode. For example, if the intermediate sensing electrode does not detect the propagation of a stimulating electrical signal, it is likely that the stimulating electrode needs to be repositioned or otherwise inspected for malfunction. Similarly, if the intermediate sensing electrode does detect propagation of the electrical signal, a technician can be assured that the stimulating electrode assemblies are properly positioned to stimulate the targeted nerve structure.

To this end, in some embodiments, power sources coupled to the stimulating electrode assemblies and sensing electrodes can include one or more digital data processors that are configured to receive readings from one or more sensing electrodes and perform this type of troubleshooting automatically. In certain embodiments, the power source can be configured to prevent an increase in stimulation signal amplitude unless the intermediate sensing electrode reports successful propagation of the stimulating electrical signal.

While the devices disclosed herein have been described as designed to be disposed after a single use, they can in some embodiments be designed for multiple uses. In some embodiments, for example, the devices can be reconditioned for reuse after at least one use. Reconditioning can include any combination of the steps of disassembly of a device, followed by cleaning or replacement of particular pieces, and subsequent reassembly. In particular, a device can be disassembled, and any number of the particular pieces or parts of the device can be selectively replaced or removed in any combination. Upon cleaning and/or replacement of particular parts, a device can be reassembled for subsequent use either at a reconditioning facility or by a surgical team immediately prior to a surgical procedure. Those skilled in the art will appreciate that the reconditioning of a device can utilize a variety of techniques for disassembly, cleaning/replacement, and reassembly. Use of such techniques, and the resulting reconditioned device, are all within the scope of the present invention.

All papers and publications cited herein are hereby incorporated by reference in their entirety. One skilled in the art will appreciate further features and advantages of the invention based on the above-described embodiments. Accordingly, the invention is not to be limited by what has been particularly shown and described, except as indicated by the appended claims.

What is claimed is:

1. (canceled)

2. The assembly of claim 27, wherein the distance between the first and second electrodes of each stimulating electrode assembly is in a range of about 10 mm to about 30 mm.

3. The assembly of claim 27, wherein the distance between the first and second electrodes of each stimulating electrode assembly is about 22 mm.

4. The assembly of claim 27, wherein the first and second electrodes of each stimulating electrode assembly have a length in a range of about 25 mm to about 40 mm.

5. The assembly of claim 27, wherein the non-conductive substrate of each stimulating electrode assembly has first and second opposed surfaces, and wherein an adhesive is disposed on the first surface, and the first and second electrodes are mounted on the first surface.

6. The assembly of claim 27, wherein the first electrode of each stimulating electrode assembly is configured as a cathode and the second electrode of each stimulating electrode assembly is configured as an anode.

7. (canceled)

8. (canceled)

9. (canceled)

10. (canceled)

11. The assembly of claim 27, wherein the impedance of the first and second electrodes of each stimulating electrode assembly is in a range of about 0 Ohms to about 10,000 Ohms.

12. The assembly of claim 29, wherein the first and second porous pads of each stimulating electrode assembly are each formed of reticulated foam.

13. The assembly of claim 29, wherein the first and second porous pads of each stimulating electrode assembly are each formed of a foam having a porosity in a range of about 10 pores per inch to about 90 pores per inch.

14. (canceled)

15. An electrode assembly configured to deliver electrical stimulation to nerves on opposing extremities of a patient's body to detect differences in evoked potentials developed in the patient's brain, comprising:
a plurality of flexible substrates configured to adhere to a tissue surface, each substrate having:
a width greater than a length thereof;
a first electrode disposed on the substrate, the first electrode including a first porous pad having a width dimension extending along the width of the substrate that is greater than a length dimension extending along the length of the substrate; and
a second electrode disposed on the substrate and spaced a distance apart from the first electrode, the second electrode including a second porous pad having a width dimension extending along the width of the substrate that is greater than a length dimension extending along the length of the substrate;
wherein the first and second porous pads on each substrate have any of a surface area and a shape effective to allow targeting of a nerve with electrical pulses when placed such that a nerve extends between the first and second porous pads at any position along the width dimension thereof;
wherein each substrate is configured to be adhered to a patient such that the length is substantially parallel to the nerve being stimulated.

16. The assembly of claim 15, wherein the first and second porous pads each have an elongate rectangular shape and extend substantially parallel to each other.

17. The assembly of claim 15, wherein the distance between the first and second porous pads is about 22 mm.

18. The assembly of claim 15, wherein the first and second electrodes of each substrate include a conductive button positioned between the substrate and the porous pad for delivering energy to the porous pad.

19. The assembly of claim 15, wherein the first and second porous pads each include a conductive sheet positioned between the substrate and the porous pad for delivering energy to the porous pad.

20. The assembly of claim 15, wherein the first and second porous pads are impregnated with a conductive gel.

21. (canceled)

22. (canceled)

23. (canceled)

24. The method of claim 32, wherein the first electrode assembly is affixed to a patient's right wrist and the second electrode assembly is affixed to a patient's left wrist to stimulate a right median nerve and a left median nerve upon delivery of the electrical signal.

25. The method of claim 32, wherein the first electrode assembly is affixed to a patient's right knee and the second electrode assembly is affixed to a patient's left knee to stimulate a right common peroneal nerve and a left common peroneal nerve upon delivery of the electrical signal.

26. The method of claim 33, further comprising coupling a connector formed at a proximal end of the ribbon cable to the energy source.

27. An apparatus for delivering electrical stimulation to nerves on opposing extremities of a patient's body to detect differences in evoked potentials developed in a patient's brain, comprising:
a first stimulating electrode assembly and a second stimulating electrode assembly, each including a non-conductive substrate having a first electrode and a second electrode mounted thereon and configured to deliver stimulating energy to an extremity of a patient's body, the first and second electrodes of each assembly having an elongate substantially rectangular shape and being spaced a distance apart from one another; and
a ribbon cable formed from a plurality of lead wires coupled together along a length thereof, each lead wire being electrically insulated from other lead wires, coupled to one of the first and second, stimulating electrode assemblies, and configured to be selectively separable from the ribbon cable;
wherein the first stimulating electrode assembly is configured to be affixed to a first extremity on a first side of the patient's body and the second stimulating electrode assembly is configured to be affixed to a second extremity on a second, opposite side of the patient's body.

28. The apparatus of claim 27, wherein the distance between the first and second electrodes of each stimulating electrode assembly is sufficient to prevent electrical shorting between the first and second electrodes.

29. The apparatus of claim 27, wherein the first and second electrodes of each stimulating electrode assembly include a porous pad impregnated with a conductive gel.

30. The assembly of claim 15, wherein the first electrode of each substrate is configured as a cathode and the second electrode of each substrate is configured as an anode.

31. A method for stimulating nerves to detect differential brain function, comprising:
affixing a substrate to a first extremity on a first side of the patient's body such that a nerve extends under the substrate along a length thereof and first and second electrodes mounted on the substrate are positioned in contact with the skin surface over the nerve;
delivering electrical energy to the nerve through the first and second electrodes;
detecting a potential in the patient's brain evoked in response to delivering the electrical energy; and
comparing the detected potential to a second potential detected in response to stimulation of a second extremity on a second, opposite side of the patient's body.

32. A method for delivering electrical stimulation to nerves on opposing extremities of a patient's body to detect differences in evoked potentials developed in the patient's brain, comprising:
affixing a first electrode assembly adjacent to a nerve to be stimulated on a right extremity of a patient's body;
affixing a second electrode assembly adjacent to a nerve to be stimulated on a left extremity of the patient's body; and
activating an energy source to deliver an electrical signal to the first and second electrode assemblies.

33. The method of claim 32, further comprising:
separating the first electrode assembly from a plurality of electrode assemblies by drawing a lead wire coupled to the first electrode assembly away from a ribbon cable that includes a plurality of lead wires coupled to the plurality of electrode assemblies and coupled to one another along a length thereof; and
separating the second electrode assembly from the plurality of electrode assemblies by drawing a lead wire coupled to the second electrode assembly away from the ribbon cable.

34. The method of claim 32, further comprising
detecting propagation of the electrical signal along a nerve chain at an intermediate location between the patient's brain and the first electrode assembly.

35. The method of claim 34, further comprising preventing an increase in an amplitude of the electrical signal if propagation of the electrical signal has not been detected at the intermediate location.
